(19) United States
(12) Patent Application Publication
(10) Pub. No.: US 2022/0009764 A1
(43) Pub. Date: Jan. 13, 2022

(54) MICRON-RESOLUTION SOFT STRETCHABLE STRAIN AND PRESSURE SENSOR

(71) Applicant: THE REGENTS OF THE UNIVERSITY OF CALIFORNIA, Oakland, CA (US)

(72) Inventors: Yongxiao Zhou, Irvine, CA (US); Michael Chu, Irvine, CA (US); Thao Nguyen, Irvine, CA (US); Michelle Khine, Irvine, CA (US); Erik Morgan Werner, Irvine, CA (US); Elliot En-Yu Hui, Irvine, CA (US); Eugene Lee, Irvine, CA (US); Kevin Costa, Irvine, CA (US)

(21) Appl. No.: 17/369,658

(22) Filed: Jul. 7, 2021

Related U.S. Application Data
(60) Provisional application No. 63/048,997, filed on Jul. 7, 2020.

(51) Int. Cl. B81B 3/00; B01L 3/00; B81B 7/02; B81C 3/00

(52) U.S. Cl. CPC B81B 3/0027; B01L 3/502707; B81B 7/02; B81B 2201/0292; B01L 2300/0627; B81B 2201/06; B81C 3/001

(57) ABSTRACT

The present invention features a stretchable strain sensor for detecting minute amounts of strain or pressure. The stretchable strain sensor may comprise a first soft polymer layer, a wrinkled conductive layer disposed on the first soft polymer layer, and a second soft polymer layer disposed on the wrinkled conductive layer. Strain applied to the sensor may cause the wrinkled conductive layer to stretch and crack and send a signal based on resistance. Pressure applied to the sensor may cause the wrinkled conductive layer to deform and crack and send a signal based on resistance. The stretchable strain sensor may be capable of measuring contractions of a tissue, detecting fluid flowing through a microfluidic channel, and detecting whether a microfluidic valve is closed or not.

### CROSS-REFERENCES TO RELATED APPLICATIONS

This application is a non-provisional and claims benefit of U.S. Provisional Application No. 63/048,997 filed Jul. 7, 2020, the specification of which is incorporated herein in their entirety by reference.

### FIELD OF THE INVENTION

The present invention is directed to a sensor capable of in vitro organoid movement detection, microfluidic flow and pressure detection, and real time monitoring of valve status in microfluidic chips.

### BACKGROUND OF THE INVENTION

Microfluidic devices for various applications, including molecular analysis, cellular analysis, and drug screening, require precise control of parameters such as pressure and flow rate. Fluid delivery is typically accomplished using off-chip hardware including pressure regulators for pressure driven flow and syringe pumps to control volumetric flow. While routing and switching of fluids can be accomplished on-chip using integrated valves, they are ultimately controlled by external pressure sources and solenoids. Feedback from these systems, including parameters such as pressure or flow rate, are typically provided by sensors off-chip, located either in the tubing connected to the device or integrated into the perfusion hardware.

Despite tremendous advances of micro total analysis systems in recent years, widely accessible on-chip monitoring and closed loop control of fundamental parameters are still lacking. The dearth of on-chip monitoring solutions creates inherent limitations in the responsiveness and accuracy of the measurements that can be obtained. Off-chip hydraulic and pneumatic sensors are limited by the dead volume of the interface tubing connecting the sensors to the chip. This dead volume is typically large compared to the volume of the microfluidic device itself and can be the dominant factor in determining the response time and accuracy of a measurement.

Currently available options for local measurement of these parameters are difficult to integrate into microfluidic systems. While optical sensors can produce accurate, reliable, and robust flow and pressure measurements, they still require coupling to expensive and complicated imaging systems. Micro electromechanical systems (MEMS)-based sensors offer on-chip integration with high resolution but typically involve complex fabrication and contact-based measurements. For example, in-channel sensors that extend into the fluid channel affect the local flow profile and can suffer from confounding factors including fouling; increased drag force from fouling can cause inaccurate results 14. Commercially available MEMS sensors are not intended for single-use applications unlike microfluidic devices; hence this mismatch in cost and complexity has prevented more pervasive integration.

Soft, stretchable sensors have attracted research interest due to their ability to conform to different surfaces and their large dynamic range under deformation. These sensors convert mechanical displacement into electrical signals such as resistance or capacitance change. Liquid metal-based pressure sensors with a polydimethylsiloxane (PDMS) substrate can be easily integrated into microfluidic devices. However, channels that contain liquid metal require extra precautions during fabrication or are more prone to mechanical failure. Alternatively, thin metal film-based sensors are easier and safe to fabricate and handle and offer attractive performance and robustness characteristics. Due to their physical properties, these metal thin film based sensors are able to sense mechanical deformations in various planes; the resulting electrical signals can be correlated and calibrated to physical parameters-of-interest. However, these soft strain gauges have been typically limited to microscale applications. There are few reports of soft sensors capable of monitoring micro-scale strains. Even recent papers focused on micron scale sensors still report monitoring deformations on the millimeter scale.

The ability to monitor deformations from extremely small forces require unique strategies. For instance, wearable sensors may not respond as linearly in this micro-regime as in macro-level, and gauge factor has been reported to be different between low strain range and high strain range. Secondly, in micro-applications, the system may not be able to actuate the strain sensor due to limited force output (e.g. the small force generated from a monolayer of cardiomyocytes, or small pressure changes in a microfluidic channel). The stress generated by an isolated muscle strip ranges from 8 to 20.7 kPa, which is not strong enough to drive conventional rigid force gauges.

To date, there are a limited number of works that have demonstrated the effective application of flexible sensors in micro-device monitoring. Parker and colleagues developed a high-sensitivity piezoresistive sensor using multi-material 3D printing to monitor stress induced by cardiac tissues, with a reported minimum tested strain of 0.0125%. Flexible sensors such as this have the potential to replace traditional optical methods to monitor tissue contractility. Wen and colleagues developed a silver powder doped-PDMS based piezoresistive pressure sensor that can be bonded to a microfluidic device. When the pressure in the channel increases, the flexible sensor is stretched.

In situations of pressure driven flow, the pressure is directly proportional to flow rate. Thus, the flow rate can be calculated from the pressure measured by a sensor in the fluid channel. While most reported non-contact flow meters have a resolution of tens to hundreds of μl/min, some research groups have demonstrated nanoliter resolution temperature flow sensors and 0.5 μl/min resolution microwave flow sensors. However, temperature flow sensors could be disturbed by non-flow effects, such as environmental heat flux flowing into sensors during experiments. Unlike other parameters, pressure is still a flow indicator that is independent from surrounding noise such as electromagnetic waves and heat flux. In the flow sensor by Sanati-Nezhad and colleagues, pressure in a microfluidic channel deforms a membrane to modulate the permittivity of a microwave resonator, thus producing a flow measurement.

Current microphysiological systems (MPS) are a good way to simulate the in vitro behavior of tissue and organs and help understand the complexity of in vivo behavior, Cardiomyocyte's contractile stress, specifically, is studied a lot due to its importance to cardiovascular disease. It is necessary to use a long term and reliable method to monitor the contractile stress.

Current methods of detecting contractile stress of cardiomyocyte includes optical tracking, which optically tracks the deflection of the substrate material that supports cardiomyocyte tissue, and electronic tracking, which uses a soft strain sensor to measure the curvature of the bended substrate material supporting the tissue. Optical method is suitable for short term studies but not very good for long term use. The analysis of optical methods involves heavy image analysis. Current electronic tracking method replaces the microscope that was used to track the deflection. Instead, electronic strain sensors are used to measure the bending curvature of the substrate. However, this is still not a direct way to measure stress because it measures the bending curvature and calculates out the stress.

The second problem is how to measure pressure and flow rate inside the channel for current microfluidic devices. Current commercialized devices can measure the pressure and flow rate inside the inlet or outlet but not inside the channel. Additionally, those devices are expensive and hard to be embedded into microfluidic chips.

Another problem is how to monitor valve status in microfluidic chips. Currently, only high-speed cameras could be used to monitor the opening and closing of valves. This requires extra image processing and time. Alternatively, sensors can be embedded into the microfluidic devices to monitor the pressure; however, they are not capable of being stretched or measuring tensile stress. A sensor that can electrically monitor valve status as well as measure both tensile stress and channel pressure is ideal.

From the current literature on available sensors for micron scale in-situ monitoring, there remains the need to develop a universal sensor compatible with soft lithography that can be scaled, arrayed, and used to measure a range of critical microfluidic parameters

### BRIEF SUMMARY OF THE INVENTION

It is an objective of the present invention to provide devices and methods that allow for in vitro organoid movement detection, microfluidic flow and pressure detection, and real time monitoring of valve status in microfluidic chips, as specified in the independent claims. Embodiments of the invention are given in the dependent claims. Embodiments of the present invention can be freely combined with each other if they are not mutually exclusive.

The present invention features an encapsulated wrinkled conductive thin film based flexible piezoresistive sensor with tunable elastic modulus that can measure micron-scale strain, microfluidic device pressure, and valve state. This soft strain sensor has a dynamic range of 50% and can detect linear displacements as small as 5 μm (0.025% strain). The displacement of the sensor can be used to calculate the force applied to the sensor. Due to its high strain sensitivity to linear stretching and ultra-soft substrate, small pressures applied on the surface deform the sensor, causing it to expand orthogonally to serve as a highly sensitive pressure sensor for microfluidic applications. The pressure measured from microfluidic devices can be correlated to flow rate in the channel as well. Finally, the sensor can be integrated into a pneumatic valve to monitor valve actuation. To the best of the inventors' knowledge, there is no such sensor that can electrically monitor valve state in microfluidic devices.

In some aspects, the present invention features a stretchable strain sensor for detecting strain and deformation. The sensor may comprise a first soft polymer layer, a wrinkled conductive layer disposed on the first soft polymer layer, and a second soft polymer layer disposed on the wrinkled conductive layer. Strain applied to the sensor may cause the wrinkled conductive layer to stretch and crack, thus sending a signal based on the resistance. Pressure applied to the sensor may cause the wrinkled conductive layer to deform and crack, thus sending a signal based on the resistance. The sensor may detect both small force and pressure. The sensor may be used for detecting tissue contractions, detecting fluid directed through a microfluidic channel, or whether or not a microfluidic valve is closed or not.

The present invention features a method for measuring strain using a stretchable strain sensor. The method may comprise providing the stretchable strain sensor comprising a first soft polymer layer, a wrinkled conductive layer disposed on the first soft polymer layer, and a second soft polymer layer disposed on the wrinkled conductive layer. The method may further comprise applying strain to the sensor, stretching and cracking, by the wrinkled conductive layer, in response to the strain on the sensor, generating, by the wrinkled conductive layer, resistance as a result of stretching and cracking, and sending a signal based on the resistance generated by the wrinkled conductive layer.

The present invention features a method for measuring pressure using a stretchable strain sensor. The method may comprise providing the stretchable strain sensor comprising a first soft polymer layer, a wrinkled conductive layer disposed on the first soft polymer layer, and a second soft polymer layer disposed on the wrinkled conductive layer. The method may further comprise applying pressure to the second soft polymer layer, stretching and cracking, by the wrinkled conductive layer, in response to the strain on the sensor, generating, by the wrinkled conductive layer, resistance as a result of stretching and cracking, and sending a signal based on the resistance generated by the wrinkled conductive layer.

The super sensitive stretchable strain sensor can be embedded into current in vitro MPS. It measures uniaxial force (as low as 20 micro-N) directly and outputs electronic reading continuously. It does not require complex mathematical calculation or numerous image processing. The sensor is also capable of measuring pressure that is applied on it, and this can be leveraged for in-channel pressure detection in microfluidic chips. The sensor is also able to be optimized and embedded in microfluidic chips as part of the valve so that it is able to monitor valve open and closure status

One of the unique and inventive technical features of the present invention is the implementation of a first and second polymer layer with an elastic modulus of 225 to 275 kPa to increase sensitivity of the wrinkled conductive layer to stretch and crack. Without wishing to limit the invention to any theory or mechanism, it is believed that the technical feature of the present invention advantageously provides for the ability to efficiently measure minute amounts of displacement applied to the sensor by measuring resistance of the wrinkled conductive layer. None of the presently known prior references or work has the unique inventive technical feature of the present invention.

Another one of the unique and inventive technical features of the present invention is the implementation of a wrinkled conductive layer to increase the detection of strain without sacrificing overall sensitivity. Without wishing to limit the invention to any theory or mechanism, it is believed that the technical feature of the present invention advantageously provides for the ability to efficiently measure minute amounts of strain applied to the sensor by measuring resistance of the wrinkled conductive layer. None of the presently known prior references or work has the unique inventive technical feature of the present invention. Furthermore, this inventive technical feature is counterintuitive. The reason that it is counterintuitive is because the technical feature contributed to a surprising result. Wrinkled features in strain sensors are well known in the art to increase the detection of strain alone, but decrease the overall sensitivity of the sensor with regards to displacement, force, etc. One skilled in the art would not implement wrinkled features in sensors for measuring incredibly small amounts of displacement due to the reduced sensitivity that comes with it. Surprisingly, the tuning of the elastic modulus of the polymer layers is able to cancel out and even overcome the reduced sensitivity of the sensor from the wrinkled conductive layer while maintaining the increased strain detection gained from the said wrinkled conductive layer. Thus, the inventive feature of the present invention contributed to a surprising result and is counterintuitive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

This patent application contains at least one drawing executed in color. Copies of this patent or patent application publication with color drawing(s) will be provided by the office upon request and payment of the necessary fee.

The features and advantages of the present invention will become apparent from a consideration of the following detailed description presented in connection with the accompanying drawings in which:

FIG. 1A shows a diagram of a stretchable strain sensor of the present invention.

FIG. 1B shows a diagram of the stretchable strain sensor of the present invention with cross-sectional dimensions of the sensor. The functional metal layer is sandwiched in between two layers of PDMS.

FIG. 2 shows a flow chart of detecting strain using the stretchable sensor of the present invention.

FIG. 3 shows a flow chart of detecting pressure using the stretchable sensor of the present invention.

FIG. 4 shows a flow chart of detecting strain, pressure, or deformation using the stretchable sensor of the present invention.

FIG. 5 The fabrication process of the microfluidic chip. A one-sided adhesive film (red, ˜50 μm thickness) is put on top of a piece of acrylic. A laser etches the channel design and removes unnecessary parts. A piece of acrylic frame is pressed and the edges are glued. The mold is filled with PDMS. After cured, the PDMS chunk is taken out. The adhesive film is removed from the PDMS and holes are punctured at the inlet and outlet position. Plasma is used to treat the PDMS chunk and the sensor, then press together. The result is a bonded sensor and microfluidic channel.

FIG. 6A shows a photograph of a stretchable strain sensor used for detecting contractions of a tissue.

FIG. 6B shows a photograph of a stretchable strain sensor used for detecting fluid running through a microfluidic channel.

FIG. 6C shows a photograph of a stretchable strain sensor for detecting whether a microfluidic valve is open or closed.

FIG. 7A shows unstretched (top) and stretched (bottom) sensors. On the right are scanning electron microscope images of sensor trace regions. It is apparent on the SEM that the wrinkles align and stress in the direction of actuation. Fractures in the thin film have been illustrated by pseudo-coloring the exposed polymer layer in red.

FIG. 7B shows the sensors resistance response under different cyclic frequencies (from top to bottom are 0.5, 1, and 2 Hz respectively). The sensor (blue) tracks the displacement of 5 μm (red) very well, and the baseline shift is captured for 2 Hz as well (˜265 s). 5 μm corresponds to 0.025% strain of the sensor.

FIG. 7C shows a representative sensitivity curve plotting change in resistance as a function of length up to 150 μm, with inset highlighting the 0 to 50 μm range; sensitivity increases with greater stretch non-linearly.

FIG. 7D shows a response test of a representative sensor, the blue line represents the sensors resistance change while the red line represents the relative change of the position of the actuator. The sensor is stretched by 200 μm within 1 second. The mean latency between sensors and actuator is 50 ms, (n=10).

FIG. 8A shows a picture of a microfluidic device with an embedded sensor.

FIG. 8B shows a schematic cross-section of the device.

FIG. 8C shows pressure sensitivity of the sensor, when the sensor is compressed 5 times. Blue line is the actual sensor sensitivity curve with red bars as standard error at each 2 kPa increments, and the yellow and purple lines are the linear fitting lines corresponding to 0-12 and 12-30 kPa pressure ranges. R2 values of 0.941 is achieved for 0-12 kPa range, and 0.987 is achieved for 12-30 kPa range.

FIG. 8D shows pressure and sensor data for flow rate increases from 0 to 50 μl/min in 10-μl/min increments, and repeated 3 times.

FIG. 8E shows a change of pressure vs. change of resistance as flow rate increases from 0 to 200 μl/min.

FIG. 8F shows post-processed sensor resistance and pressure tracings for 10 cycles of flow rate from 0 to 20 μl/min. The processed sensor signal decay stabilizes after 3 cycles.

FIG. 9A shows a sensor integrated into an elastomeric membrane valve for control of reagent flow. White scale bar is 1 cm.

FIG. 9B shows valve construction details. Close up view of overhead (top inset) and cross-section of valve (bottom inset). Channels on the control and flow layers are shown in red and blue, respectively. Sensor is embedded in the membrane shown in green. Valve seat length (L)=1.2 mm, valve seat width (W)=2.4 mm, membrane thickness (T)=0.6 mm.

FIG. 9C shows how sensor resistance increases when the valve is opened or closed.

FIG. 9D shows a sensor integrated into a microfluidic inverter logic gate for microfluidic computing.

FIG. 9E shows inverter gate construction details. Channels on the control and flow layers are shown in red and blue, respectively. Sensor placement shown in green.

FIG. 9F shows a comparison of sensor resistance and inverter output over time.

FIG. 9G shows a photo of a microfluidic oscillator pump with an integrated sensor. Light reflected from a single valve was used for high speed video analysis (inset). Only the sensor on the left is used.

FIG. 9H shows a schematic of the peristaltic pump controlled by an integrated ring oscillator circuit. The sensor was placed under the final pump valve to detect opening and closing.

FIG. 9I shows a comparison of sensor data and high-speed video for monitoring oscillation frequency showing matching peaks at 6.71 Hz.

FIG. 9J shows a flow rate from the peristaltic pump measured using external hot wire anemometer and corresponding sensor measurements from the final valve in the pump. All scale bars are 1 cm.

FIG. 10 shows a graph of hysteresis of the sensor under loading and unloading for 20 times. It is clear that the sensor follows different trajectories when loaded and unloaded.

FIG. 11 shows the channel of a microfluidic chip under no flow and no pressure, and the sensor is not deformed (left) and a deformed sensor under flow, which has higher pressure, and the sensor is deformed (right).

FIG. 12 shows simulated results of pressure drop across the length of a microfluidic channel.

FIG. 13 shows a graph of a detection limit and resolution test. Flow rate increases from 0 to 30 μl/min with 2 μl/min increment. Blue, green, and red shaded areas are three flow rate sections, which are 2, 4, and 6 μl/min. Sensor signal (blue line) starts to increase when flow rate is greater than 6 μl/min.

FIG. 14 shows sensing of the pump pattern generated by an oscillator pump. As pressure changes propagate through the ring oscillator, pump valves are opened and closed in a peristaltic pumping pattern. A sensor embedded pump valve detects changes in valve state that correspond to the instantaneous flow rate from the pump. A backward flow pulse occurs when valve 3 opens followed by two forward flow pulses that occur when valves 2 and 3 close.

FIG. 15 shows monitoring of oscillator frequency. The frequency of the ring oscillator was adjusted by adding varying resistances to the air inlets of one inverter gate. Fourier transforms of the sensor measurement and video-based measurement were compared for four different resistance values and show excellent agreement.

### DETAILED DESCRIPTION OF THE INVENTION

Following is a list of elements corresponding to a particular element referred to herein:

1 stretchable strain sensor

100 first soft polymer layer

200 wrinkled conductive layer

300 second soft polymer layer

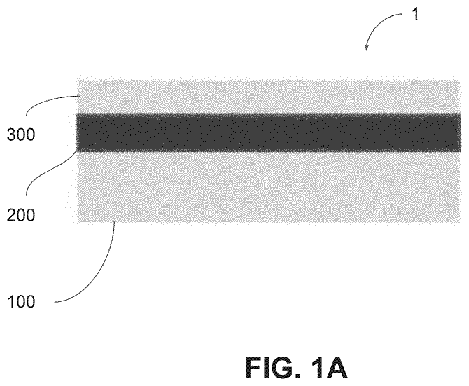

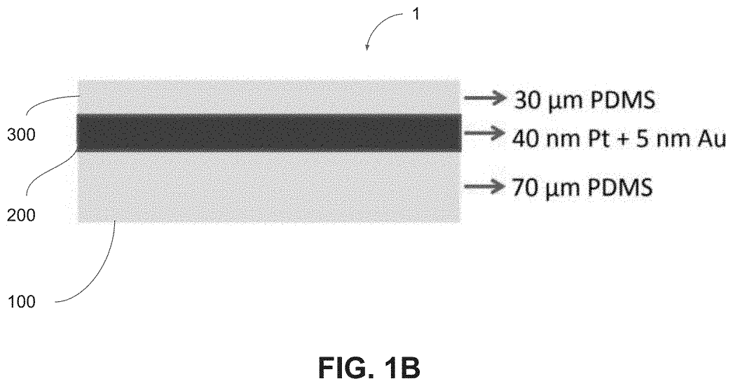

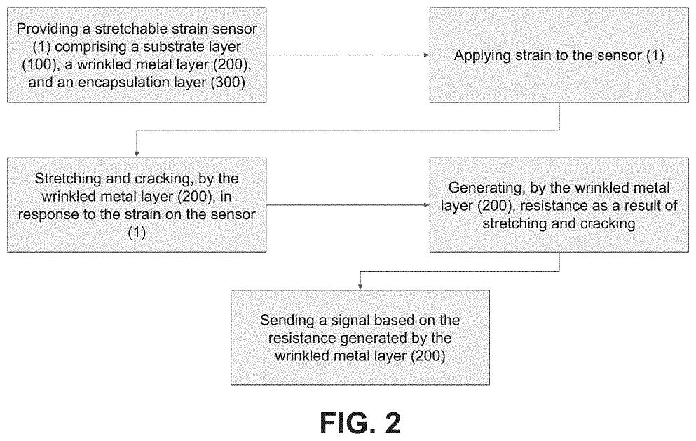

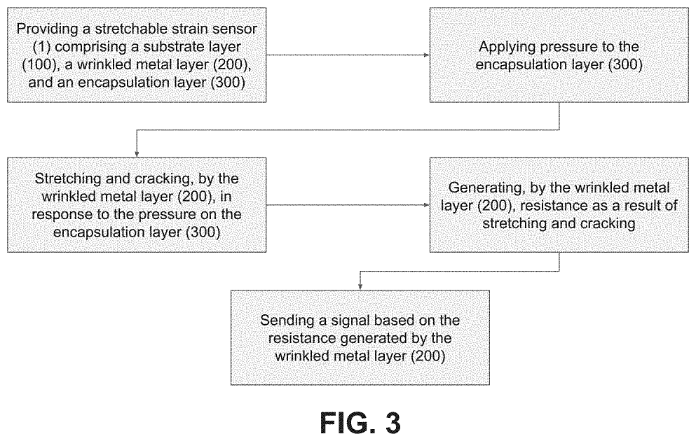

Referring now to FIGS. 1A-1B, the present invention features a stretchable strain sensor (1) for detecting strain, pressure, deformation, stress, displacement, or a combination thereof. In some embodiments, the sensor may comprise a first soft polymer layer (100), a wrinkled conductive layer (200) disposed on the first soft polymer layer (100), and a second soft polymer layer (300) disposed on the wrinkled conductive layer (200). Strain, pressure, deformation, stress, displacement, or a combination thereof applied to the strain sensor (1) may cause the wrinkled conductive layer (200) to stretch and crack, creating resistance in the wrinkled conductive layer (200) and sending a signal based on the resistance. The strain sensor (1) may be capable of detecting about 5 microns of linear displacement. In some embodiments, the stretchable strain sensor (1) may be capable of measuring and sensing in vitro behavior of tissue and organs. In some embodiments, the stretchable strain sensor (1) may have dimensions of about 20 mm by about 2 mm by about 0.1 mm. In some embodiments, the first soft polymer layer (100) may have a thickness of about 30 microns. In some embodiments, the wrinkled conductive layer (200) may have a thickness of about 45 nm. In some embodiments, the second soft polymer layer (300) may have a thickness of about 70 microns. The first and second soft polymer layers may have an elastic modulus ranging from about 225 to 275 kPa. For instance, the first and second soft polymer layers may have an elastic modulus of about 250 kPa.

In some embodiments, a tissue may be disposed on the strain sensor (1) and contractions of the tissue may apply strain to the strain sensor (1), actuating the strain sensor (1). In some embodiments, the stretchable strain sensor (1) may be capable of measuring pressure and flow rate inside a channel of a microfluidic device. In some embodiments, a microfluidic channel may be disposed on the second soft polymer layer (300) and fluid flowing through the microfluidic channel may cause pressure to be applied to the second soft polymer layer (300), actuating the sensor (1). In some embodiments, the stretchable strain sensor (1) may be capable of monitoring a status of a valve in a microfluidic device. In some embodiments, the sensor (1) may be disposed in a microfluidic valve and opening the microfluidic valve may cause deformation of the wrinkled conductive layer (200), actuating the sensor (1). In some embodiments, the first soft polymer layer (100) may comprise polydimethylsiloxane (PDMS), hydrogel, silicon-based polymers, polyurethane-based polymers, any polymer that can be molded, elastomers, or a combination thereof. In some embodiments, the wrinkled conductive layer (200) may comprise one or more metals (e.g. Au, Pd, Pt, Ag), one or more semiconductive materials (e.g. silicon), one or more nano-materials (e.g. carbon nanotubes, graphene), one or more conductive polymers (e.g. PEDOT:PSS), one or more conductive particles (e.g, silver flakes, carbon black) embedded in a polymer, or a combination thereof. The wrinkled conductive layer (200) may have a thickness of about 75 to 125 nm. In some embodiments, the wrinkled conductive layer (200) may have a thickness of about 100 nm. In some embodiments, a material of the wrinkled conductive layer (200) may determine a sensing ability of the strain sensor (1). In some embodiments, the second soft polymer layer (300) may comprise PDMS, hydrogel, silicon-based polymers, polyurethane-based polymers, any polymer that can be molded, elastomers, or a combination thereof. In some embodiments, the stretchable strain sensor (1) can be tuned to detect a wider range of forces through the use of PDMS fluid. In some embodiments, the strain sensor (1) may be capable of returning to a resting state from strain, pressure, deformation, stress, displacement, or a combination thereof in about 5-10 ms, In some embodiments, a soft polymer composition of the first and second soft polymer layers may comprise polydimethylsiloxane (PDMS) having a mass ratio of about 1-4 cure to 15-20 base to 4-5 silicone fluid. In some embodiments, the curing agent may comprise a silicone elastomer. In some embodiments, the base may comprise a silicone elastomer.

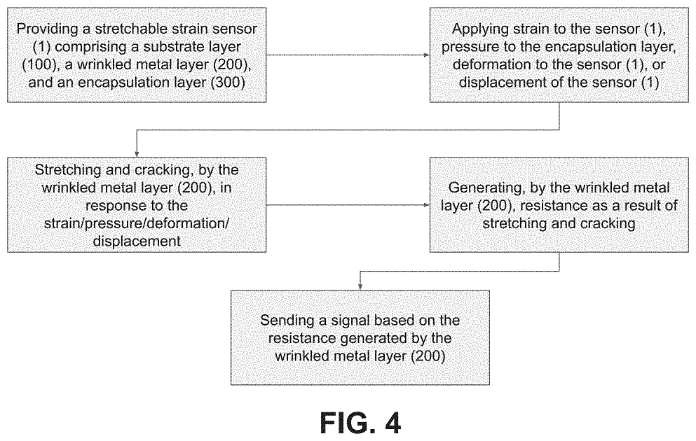

Referring now to FIG. 4, the present invention features a method for measuring strain, pressure, deformation, stress, displacement, or a combination thereof using a stretchable strain sensor (1). In some embodiments, the method may comprise providing the stretchable strain sensor (1). The sensor (1) may comprise a first soft polymer layer (100), a wrinkled conductive layer (200) disposed on the first soft polymer layer (100), and a second soft polymer layer (300) disposed on the wrinkled conductive layer (200). Strain, pressure, deformation, stress, displacement, or a combination thereof applied to the strain sensor (1) may cause the wrinkled conductive layer (200) to stretch and crack, creating resistance in the wrinkled conductive layer (200) and sending a signal based on the resistance. The strain sensor (1) may be capable of detecting about 5 microns of linear displacement. The method may further comprise applying strain, pressure, deformation, stress, displacement, or a combination thereof to the strain sensor (1), stretching and cracking, by the wrinkled conductive layer (200), in response to the strain, pressure, deformation, stress, displacement, or combination thereof of the strain sensor (1), generating, by the wrinkled conductive layer (200), resistance as a result of stretching and cracking, and sending a signal based on the resistance generated by the wrinkled conductive layer (200). The first and second soft polymer layers may have an elastic modulus ranging from about 225 to 275 kPa. The first and second soft polymer layers may have an elastic modulus of about 250 kPa.

In some embodiments, a tissue may be disposed on the strain sensor (1) and contractions of the tissue may apply strain to the strain sensor (1), actuating the strain sensor (1). In some embodiments, the stretchable strain sensor (1) may be capable of measuring pressure and flow rate inside a channel of a microfluidic device. In some embodiments, a microfluidic channel may be disposed on the second soft polymer layer (300) and fluid flowing through the microfluidic channel may cause pressure to be applied to the second soft polymer layer (300), actuating the sensor (1). In some embodiments, the stretchable strain sensor (1) may be capable of monitoring a status of a valve in a microfluidic device. In some embodiments, the sensor (1) may be disposed in a microfluidic valve and opening the microfluidic valve may cause deformation of the wrinkled conductive layer (200), actuating the sensor (1).

In some embodiments, the first soft polymer layer (100) may comprise PDMS, hydrogel, silicon-based polymers, polyurethane-based polymers, any polymer that can be molded, elastomers, or a combination thereof. In some embodiments, the wrinkled conductive layer (200) may comprise one or more metals (e.g. Au, Pd, Pt, Ag), one or more semiconductive materials (e.g, silicon), one or more nano-materials (e.g. carbon nanotubes, graphene), one or more conductive polymers (e.g. PEDOT:PSS), one or more conductive particles (e.g. silver flakes, carbon black) embedded in a polymer, or a combination thereof. The wrinkled conductive layer (200) may have a thickness of about 75 to 125 nm, In some embodiments, the wrinkled conductive layer (200) may have a thickness of about 100 nm. In some embodiments, a material of the wrinkled conductive layer (200) may determine a sensing ability of the strain sensor (1).

In some embodiments, the second soft polymer layer (300) may comprise PDMS, hydrogel, silicon-based polymers, polyurethane-based polymers, any polymer that can be molded, elastomers, or a combination thereof. In some embodiments, the stretchable strain sensor (1) can be tuned to detect a wider range of forces through the use of PDMS fluid. In some embodiments, the strain sensor (1) may be capable of returning to a resting state from strain, pressure, deformation, stress, displacement, or a combination thereof in about 5-10 ms. In some embodiments, a soft polymer composition of the first and second soft polymer layers may comprise polydimethylsiloxane (PDMS) having a mass ratio of about 1-4 cure to 15-20 base to 4-5 silicone fluid.

The present invention features a method for fabricating a stretchable strain sensor (1) into a microfluidic channel to allow measurement of strain, pressure, deformation, stress, displacement, or a combination thereof in the microfluidic channel. In some embodiments, the method may comprise depositing conductive material onto a mold, applying heat to the conductive material causing shrinkage in order to produce a wrinkled conductive layer (200), placing the wrinkled conductive layer (200) in a solution, removing the wrinkled conductive layer (200) from the solution, and rinsing the solution from the wrinkled conductive layer (200). The method may further comprise preparing and tuning a polymer composition to have an elastic modulus to 225 to 275 kPa, thereby producing a soft polymer composition. In some embodiments, the soft polymer composition has an elastic modulus of about 250 kPa The method may further comprise applying a first soft polymer layer (100) comprising the soft polymer composition to the wrinkled conductive layer (200), curing the first soft polymer layer (100) and the wrinkled conductive layer (200), removing the mold from the wrinkled conductive layer (200), and applying a second soft polymer layer (300) comprising the soft polymer composition to the wrinkled conductive layer (200) such that the wrinkled conductive layer (200) is disposed between the first soft polymer layer (100) and the second soft polymer layer (300). The strain sensor (1) may be capable of detecting about 5 microns of linear displacement.

In some embodiments, a tissue may be disposed on the strain sensor (1) and contractions of the tissue may apply strain to the strain sensor (1), actuating the strain sensor (1). In some embodiments, the stretchable strain sensor (1) may be capable of measuring pressure and flow rate inside a channel of a microfluidic device. In some embodiments, a microfluidic channel may be disposed on the second soft polymer layer (300) and fluid flowing through the microfluidic channel may cause pressure to be applied to the second soft polymer layer (300), actuating the sensor (1). In some embodiments, the stretchable strain sensor (1) may be capable of monitoring a status of a valve in a microfluidic device. In some embodiments, the sensor (1) may be disposed in a microfluidic valve and opening the microfluidic valve may cause deformation of the wrinkled conductive layer (200), actuating the sensor (1). In some embodiments, the first soft polymer layer (100) may comprise PDMS, hydrogel, silicon-based polymers, polyurethane-based polymers, any polymer that can be molded, elastomers, or a combination thereof. In some embodiments, the wrinkled conductive layer (200) may comprise one or more metals (e.g. Au, Pd, Pt, Ag), one or more semiconductive materials (e.g, silicon), one or more nano-materials (e.g, carbon nanotubes, graphene), one or more conductive polymers (e.g. PEDOTPSS), one or more conductive particles (e.g. silver flakes, carbon black) embedded in a polymer, or a combination thereof. The wrinkled conductive layer (200) may have a thickness of about 75 to 125 nm. In some embodiments, the wrinkled conductive layer (200) may have a thickness of about 100 nm. In some embodiments, a material of the wrinkled conductive layer (200) may determine a sensing ability of the strain sensor (1). In some embodiments, the second soft polymer layer (300) may comprise PDMS, hydrogel, silicon-based polymers, polyurethane-based polymers, any polymer that can be molded, elastomers, or a combination thereof. In some embodiments, the stretchable strain sensor (1) can be tuned to detect a wider range of forces through the use of PDMS fluid. In some embodiments, the strain sensor (1) may be capable of returning to a resting state from strain, pressure, deformation, stress, displacement, or a combination thereof in about 5-10 ms. In some embodiments, the solution may comprise a 5 mM 3-mercaptopropyl trimethoxysilane (MPTMS) ethanol solution. In some embodiments, the soft polymer composition may comprise polydimethylsiloxane (PDMS) having a mass ratio of about 1-4 cure to 15-20 base to 4-5 silicone fluid.

The sensor was well designed and was sensitive enough to detect ˜5 micrometer stretching. Due to the special geometry design, 5 micrometer displacement requires 20 micro-N tensile force, and a typical cardiomyocyte tissue contracted with 20 micro-N force. The larger the force was, the longer the sensor was stretched, and higher the output of the sensor was. After the initial force-displacement calibration was done, the sensor output was either displacement or force. The tip of the sensor was designed in the way that cells anchored and grew on it. The bottom of the sensor was sandwiched between two protective layers and was fixed in the desired position. Once the tissue that was attached to the tip started to contract, the sensor was stretched and the resistance of the functional metal layer in the sensor increased. The sensor consists of three layers: Polydimethylsiloxane (PDMS) substrate layer, functional metal layer (platinum and gold), and PDMS encapsulation layer. When the sensor was stretched, the wrinkled functional metal layer was stretched and formed cracks on it: therefore, the resistance of the metal layer went up.

Similarly, when there was fluid flowing across the channel in a microfluidic device that was on top of the sensor, the pressure in the channel deforms the channel wall and the bottom layer which was the encapsulation layer of the sensor. The deformation of the encapsulation layer also deforms the functional metal layer and introduces cracks on it; thus, higher the pressure in the channel, more deformation in the channel and sensor, more cracks form, higher the sensor resistance. Flow rate and pressure change were back calculated from the sensor reading.

When the sensor was embedded in the microfluidic chips as part of the valve, it deforms at different valve status. There was no deformation when the valve was closed, and the resistance of the sensor was low; when the valve was open, the deformed valve deforms the sensor, and resistance of the sensor was higher. As a result, valve open and closure status could be read from sensor resistance. Partially opened valves were detected by the sensor as well.

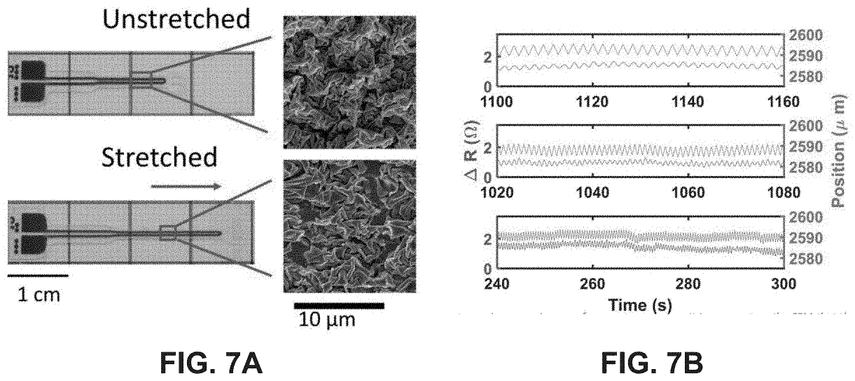

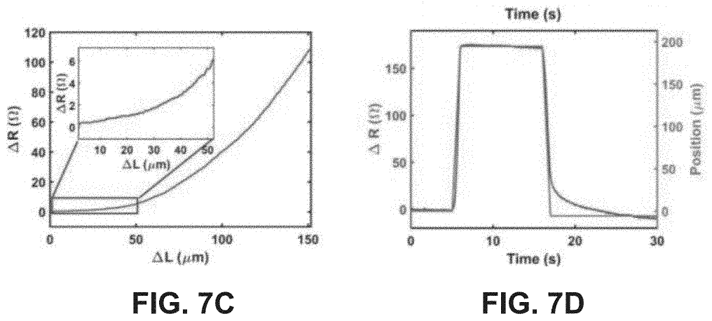

The present invention is characterized by piezoresistive sensors with integrated nano-to-micro scale wrinkled structures (FIG. 7). This thin film was supported on and encapsulated with a silicone elastomer. The resistance change of the sensors was based on crack formation within the wrinkled film when stretched. When the sensor was strained linearly, deformation of the substrate caused elongation of the metal thin film. In comparison to planar thin films, the wrinkled film allowed for a considerably larger dynamic range because the wrinkles unfolded, aligned to the axis of strain, and stretched before cracks formed. The subsequent cracking corresponded to a steeper increase in resistance as the cracks propagated and coalesced. In FIG. 7A, wrinkles were observed to stretch along the direction of the applied strain, indicated by the arrow. Cracks were formed as well. The red pseudo-color illustrates the exposed bottom polymer substrate and the cracks around it. The wrinkles acted as additional strain relief, allowing for considerably larger dynamic range, while maintaining high sensitivity, before irreversible failure.

The composition of the functional metal thin film was tuned to achieve a balance of brittleness and stability in the sensor to achieve a stretch resolution of 5 microns. The metal thin film was a bilayer of platinum and gold. Material brittleness affects the number and size of cracks that form along with the energy required to form cracks. Platinum is a more brittle material while gold has good ductility. A thicker platinum layer resulted in more and larger cracks but led to unstable resistance. As a more ductile material, a gold layer led to fewer cracks, but the change in resistance was significantly smaller. A balance was achieved by controlling the thickness of platinum and gold, respectively. After testing various combinations, a 40 nm platinum was chosen along with a 5 nm gold layer because it provided the highest signal detection while still maintaining stability. The sensor's substrate was 70 μm thick PDMS, with an encapsulation layer of 30 μm PDMS, with the wrinkled metal layer sandwiched in between the PDMS layers.

To calculate the conversion between mechanical displacement and corresponding force, certain approximations and assumptions were made. As the sensor was stretched at the micron scale with negligible deformation, the deformation of the sensor was assumed to be a uniform beam that was undergoing uniaxial stress and had elastic-like behavior. From equation 1:

σ=E·ε (1)

where σ is stress, E is Young's Modulus, and ε is strain. This is expanded in equation 2:

$$\frac{F}{w \cdot h} = E \cdot \frac{Δ L}{{L}_{0}} \qquad ( 2 )$$

where F is the uniaxial force, w and h are width and thickness of cross-sectional area, ΔL is the change in length, and L₀ is original length. Thus, to reduce the force required to actuate the sensor to displace 5 μm, the elastic modulus of the silicone substrate was lowered to 250 kPa by adding dimethyl silicone fluid (PMX 200) to a mixture of a 20:1 base-to-cure mass ratio Polydimethylsiloxane (PDMS), and substrate dimensions were adjusted to 20 mm×2 mm×0.1 mm (l×w×h). The combination of these constituents allowed the sensor to detect as low as 20 μN uniaxial force which corresponds to 5 μm linear displacement.

FIG. 7B shows a representative sensor's behavior under different stretching frequencies. It demonstrates the sensor's repeatability. The sensor's pad area was fixed on a linear actuator (Zaber Technologies Inc) with the tip area damped on a moving stage. The moving stage was cycled by 5 μm at 0.5, 1, and 2 Hz, respectively, while the sensor tracked the changes accordingly. A baseline shift of the stage movement was also captured at ˜265 s for 2 Hz. While it is not obvious in the figure, there was signal delay between the position and resistance. For 0.5, 1, and 2 Hz the response times were 169, 80, and 27 ms respectively.

To further understand the signal latency, one more experiment was performed. A typical sensor was stretched by 200 μm at a speed of 200 μm/s, held for 10 seconds, and released back by 200 μm at a speed of 200 μm/s. The position of the linear actuator and resistance of the sensor were both recorded. 34 tests (N=10 sensors) were performed. On average, the actuator began to move at 5.03±0.01 s while the sensor began to detect a resistance change at 5.08±0.08 s. The stop time was defined as the time at which the sensor or actuator reached 90% of value of the maximum relative change. The actuator stopped at 5.90±0.02 s and the sensor stopped at 5.95±0.1 s. The data indicates that the sensors have an average response time of 50 ms. Computer processing and device communication time, however, also contribute to this response time.

To observe signal hysteresis, the sensor was cycled to 150 μm and stretched at 20 μm/s speed for 20 times. From this figure, although reproducible, the sensor's resistance followed different trajectories when stretched and released at large deformations. With the loading and unloading behaviour displaying different sensitivities, it is important to know which trajectory the sensor was on when tested. As the sensors were initially stretched, wrinkles in the metallic thin film unfolded, resulting in minimal changes in resistance. As strain increases and cracks form and propagate, the resistance increases nonlinearly.

The sensor was integrated with the microfluidic chip by plasma bonding the microfluidic chip directly onto the sensor. The sensor had the same structure and design as the one used for stress-strain testing, except the PDMS substrate was larger and had not been cut into dog bone shape.

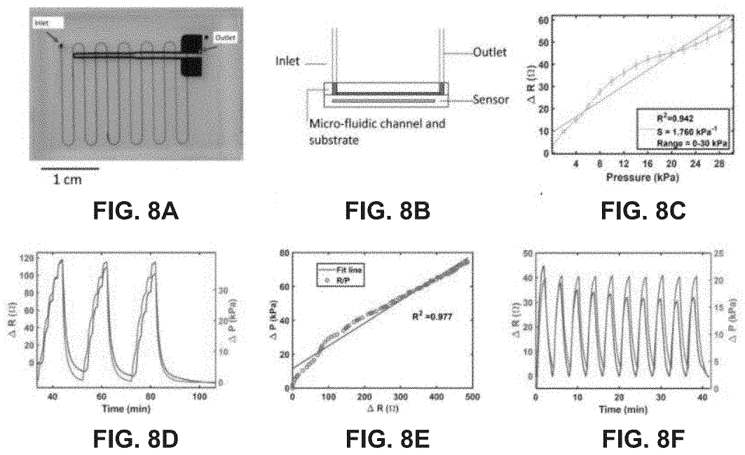

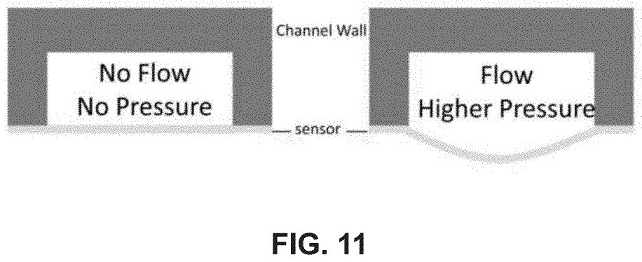

When fluid was pushed through the microfluidic device, the pressure within the channel deformed the membrane of the piezoresistive sensor (FIG. 11) and changed the electrical resistance of the functional metal film. As shown in FIG. 8A, the channel (clear, blue) overlapped with the sensing area (black).

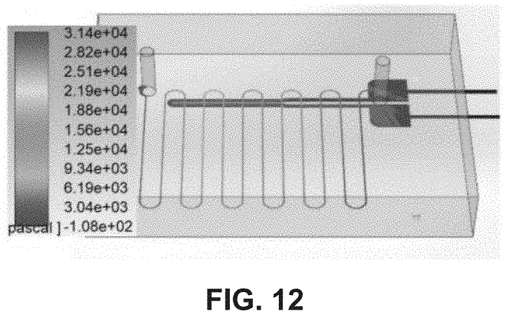

When pressure was applied normally to the sensor surface, the sensor substrate expanded in the transverse plane. Lateral expansion of the sensor elongated the metal film causing cracks to appear; when pressure was reduced from the surface, the substrate returned to its original shape, and the fractured metal came back into contact with each other. Due to the design difference between the trace and pad area, the pad area had a larger metal area. However, from the simulation results in FIG. 12, the pressure within the region that overlaps the sensor pad area was several folds smaller than that of the region overlapping the trace area.

Several aspects of the sensor performance were assessed, including working range, resolution, accuracy, and repeatability. For the microfluidic device, with a working flow rate range of 6 μl/min to 200 μl/min, the measured pressure from the inline pressure sensor of the inlet fluid varied between 1 kPa to 74 kPa. FIG. 12 depicts a simulation of pressure within the channel under a 10 μl/min flow rate. The pressure map shows the gauge pressure which was related to deformation on the channel wall and sensor. Gauge pressure inside the channel dropped along the pathway and reached 0 at the open-air outlet indicated in FIG. 8B. With a channel width of 250 μm and trace width of the sensor 300 μm, the overlap area was small in comparison to the entire sensor. The deformation of a single overlap area was too small for the signal change of the sensor to be detected. Thus, multiple sensor-channel crosses were used to increase the overlap area to boost the signal. However, from the simulation (FIG. 12), the pressure dropped along with channel length, and the deformation of the cross area became smaller with less pressure. As a result, more overlap increased the total signal sensitivity, but with diminishing returns. With the variable pressure along the channel and the sensor having multiple crosses within the channel to increase signal change, it was difficult to detect localized pressure. In this configuration, the sensor detected overall deformation caused by the pressure.

To confirm the results, a pressure sensitivity test was performed on the sensor. A 3 mm by 15 mm acrylic flat was placed over the sensor trace area. A force gauge (Mark 10 M5-025) was fixed on a test stand (Mark 10 ESM 303) and placed into contact until pressure was applied to the acrylic piece. As the pressure increased, the sensor's resistance increased as well (FIG. 8C). The blue line is the average resistance across 5 runs. Red markers indicate the standard error at every 2 kPa increment. The yellow line is the linearly fitted line, which had a R2 value of 0.942. From the graph, although the resistance value varies across sensors, they all followed the same trend and were relatively linear, especially at low pressures.

FIG. 8D represents a variable flow rate test showing pressure and sensor data versus time. Flow rate increased from 0 to 50 μl/min in 10 μl/min increments, and the entire test was repeated three times consecutively.

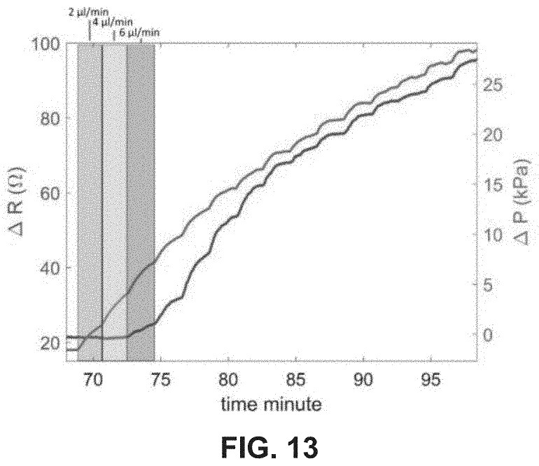

The working range for the device was 6 μl/min to 200 μl/min. The criterion for minimum resolution was that the signal change between two different flow rates was at least 3-fold larger than root mean squared noise. In a flow rate test ranging from 0 to 30 μl/min with 2 μl/min increment, data showed that the minimum detectable flow rate was 6 μl/min, and resolution was 2 μl/min (FIG. 13). Although the flow rate working range for the device was tested to an upper limit of 200 μl/min (as shown in FIG. 8E), the device was tested up to 300 μl/min without failure (data not shown).

Although sensor data showed good correlation to changes in pressure and flow rate, the baseline signal decayed when strain was removed and the sensor returned to an unstretched state. The flow rate dropped from 20 μl/min to 0 μl/min as shown in FIG. 8F. Signal decay was noticeable when the sensor reading dropped even when the linear actuator or syringe pump was idle. The decaying tails observed in FIG. 8D and supplemental. Similarly, signals at zero flow rate decreased in value as well (FIG. 8D, four separate zero flow rate points were ˜35 min, 55 min, 75 min, and 90 min). This decay complicated the data analysis and limited the duration that the resistance to pressure relationship was accurate but was accounted for with subsequent data processing. Because baseline decay occurred in all sensor data, every test data had a different baseline value. In order to compare inter-trial data with different starting baselines, all data were subtracted by the beginning baseline resistance so that it started at 0. Additionally, the decaying trend was compensated by data post-processing. As shown in FIG. 8F, the value of each valley was extracted, and set to 0 ohms. Each valley point was used to form a linear interpolated line. The data points between the valleys were adjusted by subtracting the linearly interpolated lines, between the valleys, from the signal so that the sensor signal at each zero flow rate was set to 0 Ohm.

The system elasticity was one minor issue that contributed to the signal decay; another possible contribution to the signal decay was the polymer relaxation. Relaxation was an intrinsic property of the polymer substrate. As the channel wall and sensor floor underwent mechanical hysteresis and relaxation, the formation and contact points of cracks in the embedded metal thin film were affected, resulting in an electrical hysteresis as well. Other groups have demonstrated that the hysteresis in piezoresistive based elastomeric strain sensors were potentially accounted for using machine learning.

To ensure repeatability of the sensor, conditioning tests were performed on the chip device. The fluid flowed through the pre-primed device at 20 μl/min for 2 minutes and then paused for 2 minutes; this cycle was repeated 10 times. The sensor resistance difference between 0 and 20 μl/min was compared for 10 cycles (FIG. 8F). Although some decay remains, the difference in resistance decrease was greatly reduced after 3 cycles.

The microfluidic valves used in this study were normally closed elastomeric membrane valves similar to those first reported by the Mathies group. A valve consists of two layers of microfluidic channels sandwiched around a thin elastomeric membrane. The valve was opened by applying vacuum to the control layer, deflecting the membrane and connecting the channels on the opposite side. The piezoresistive sensor was embedded in the elastomeric membrane with the sensing element placed directly over the seat of the valve, allowing the sensor to detect valve opening or closing when the sensor was stretched or relaxed.

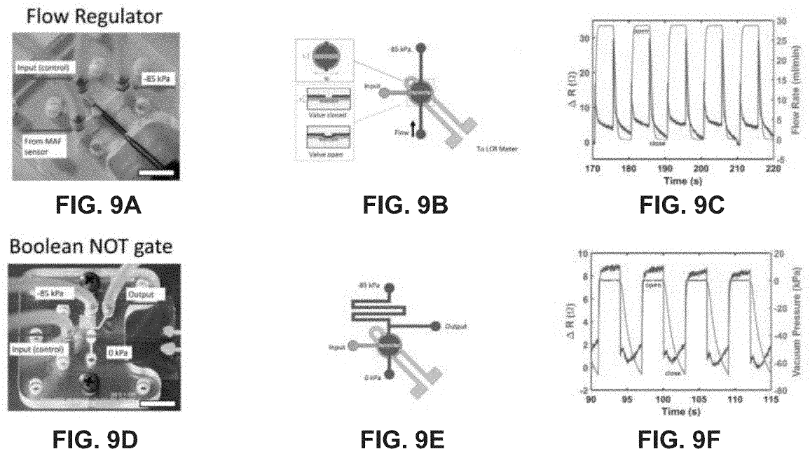

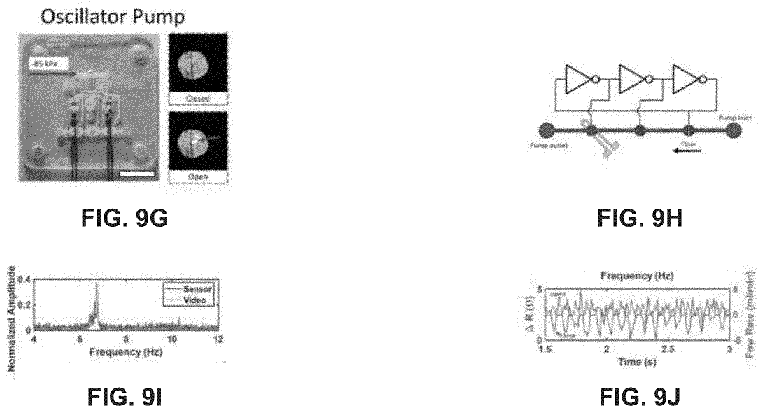

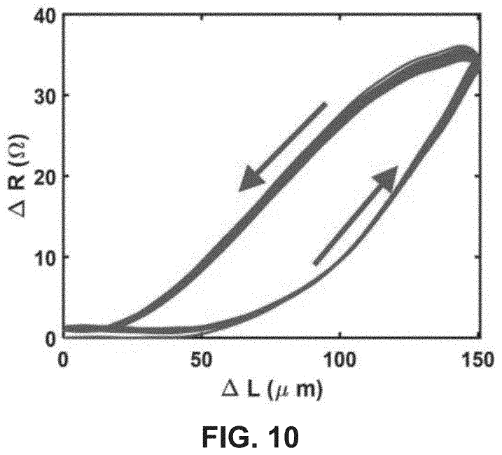

The ability of the integrated piezoresistive sensor to measure the state of a valve configured to switch fluid flow on or off (FIG. 9A) was investigated. An external hot wire anemometer (Zephyr HAF, Honeywell) was configured to measure the flow rate of air through the valve as it was opened and closed. Upon valve actuation, the integrated sensor produced a sharp spike in signal followed by an increase in baseline resistance when opened and a decrease when dosed. These results showed the membrane stretching before the valve opened followed by the membrane remaining in a partially stretched state while the valve remained open (FIG. 9C). Upon dosing, the sensor signal spiked again as the vacuum was released and the membrane contacted the valve seat sealing the valve closed. It was observed that the spike that occurred during valve state changes was dependent on the orientation of the sensor and was most pronounced when the sensor was placed directly over the seat of the valve. Data from the external air flow sensor showed the valve completely opened and closed, and the sensor did not interfere with normal operation of the valve. These results indicate the piezoresistive sensor was suitable for monitoring the state change of the valve.

Normally closed elastomeric membrane valves were also used to create digital logic gates that were well suited for building integrated microfluidic control circuitry. Therefore, the ability of the integrated piezoresistive sensor to measure the state of a valve configured as a microfluidic inverter gate was next investigated. This circuit added a pull-up resistor before the vacuum connection to the valve and an output connection upstream of the resistor to produce a digital pressure output signal that was the inverse of the input signal. The sensor reported an increase in resistance of approximately 6 ohms when the valve was opened and returned to baseline when the valve was closed, providing a clear electronic signal that corresponded to changes in the pneumatic output of the inverter gate.

Finally, to create a simple integrated microfluidic control circuit, an oscillator pump was constructed consisting of three identical inverter gates connected in a ring and three liquid handling valves, each connected to the output of an inverter gate (FIG. 9G). When a constant vacuum pressure was applied to the oscillator, the pressure sequence generated by each inverter opened and closed the pump valves to create a peristaltic pumping action. The piezoresistive sensor was placed under the final valve in the pump while high speed video imaging was used to monitor the incident light reflected when the valve membrane was pulled open (FIG. 9G inset). The oscillation frequency measured by the sensor agreed well with the measurements acquired using high speed video imaging, and the sensor was able to accurately measure oscillation frequencies as high as 24.9 Hz, approximately the Nyquist frequency of the acquisition device (FIG. 9I). Previous work has shown that the frequency of a ring oscillator can be tuned by changing the input pressure and the average flow rate of an oscillator pump was dependent on the frequency. Using this information, the oscillation frequency was used to calculate the average flow rate from the pump.

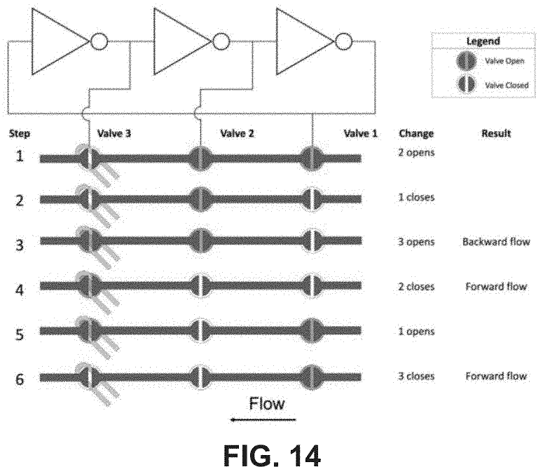

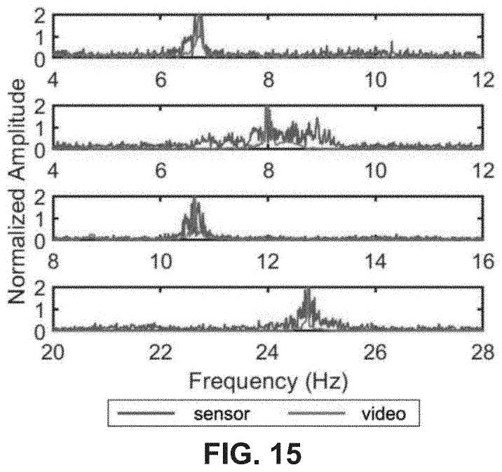

When placed under the final valve in the peristaltic pump (FIG. 9H), the sensor readings also aligned well with the pulsatile flow rate measurements acquired from a hot wire anemometer (Zephyr HAF, Honeywell) connected to the output of the pump (FIG. 9J). A small backflow was detected when the final pump valve opened that was mostly negated by the closing of the middle valve in the pump. Finally, a strong forward pulse occurred when the final valve closed. Monitoring the state of the final valve in the pump, the sensor was used to indicate the instantaneous flow rate produced by the pump. A detailed explanation of the working principle of the oscillator is provided in FIG. 14.

A soft, highly sensitive strain sensor was developed that was able to capture 5 μm linear displacement (0.025% strain for 20 mm sensor length) in the normal and uniaxial direction and was deformed with as little as 20 μN of force (100 Pa stress). The response time of the sensor for linear stretching was ˜50 ms. In comparison to other flexible pressure and strain sensor's response times, which range from ˜17 ms to ˜100 ms, the sensor shows relatively fast response.

As an indirect flow meter, the stretchable strain sensor of the present invention also detected on-site flow rate in-situ as low as 6 μl/min with a resolution of 2 μl/min in the device. However, for an embodiment with only a single sensor, failure occurred if the device was clogged. This caused the pressure to build up and the sensor readings to increase, but nothing flowed. Multiple sensors were used such that separate measurements at each intersection of the channel are acquired. This allows for more precise local pressure and flow monitoring. Moreover, it allows for the detection of clogging in the channel. As the pressure increased prior to the clogged point and decreased after the clogged point, the sensors showed relatively high or low readings at different intersections.

For monitoring microfluidic valve state, the integrated sensor provided a more direct method to monitor valve actuation than existing optical monitoring methods. The sensor monitored the binary status of a single valve precisely. Due to the analog output of the sensor, it could potentially detect partially opened valves rather than binary open and closed status; however, this may require individual calibration of each sensor.

As mentioned in the results section, hysteresis and decay of the signal affect repeatability of the sensor. With hysteresis present in the system of the present invention, only the loading signal path was used for analysis. For strain and liquid flow tests and experiments, the loading trajectory was focused on rather than unloading trajectory for consistency, particularly as the decay was less severe. For the valve experiment, the opening and closing of the valve along with other features of the actuation (such as spikes shown in FIG. 9) was qualitatively checked. Although hysteresis still exists, it was not critical here and was compensated for with the machine learning algorithms aspect for this specific application.

Due to decay, the signal baseline varied during experiments so the starting resistance was subtracted to zero the baseline for different experiments. Several attempts have been made to minimize hysteresis and decay. In one case, stiffer substrates demonstrated less decay; however, stiffer substrates required larger loads to deform which decreased the detection resolution of the sensor. For some physiological applications, it was impossible to apply larger forces. Thus, adjusting stiffness according to different applications was a potential solution to minimize hysteresis and decay. Additionally, use of other substrate materials with less intrinsic hysteresis than PDMS was possible, too.

The ability to integrate the soft and extremely sensitive strain sensor into microfluidic devices to provide contactless detection of pressure and correlation with flow rate was demonstrated. The sensor was also embedded into PDMS based valves to detect the extent of valve opening in microfluidic devices. Moreover, being PDMS-based, the sensor was easily trimmed and bonded to any other silicone-based devices via plasma treatment. The measurement results showed good linear correlation between sensor reading and flow rate and pressure in the device. The sensor had a flow rate detection range from 6 microliters per minute (μL/min) to 200 μL/min and a resolution of 2 μl/min, The sensor confirmed partial or complete valve actuation under different pressures.

Because the sensor was made of PDMS, it was compatible with soft lithography and easily integrated into microfluidic chips. The stiffness of the substrate along with the sensitivity and dimensions of the sensor can be adapted to different applications. The soft and flexible substrate also made it possible to integrate the sensor into biological applications and monitor micron-scale tissue movement. The sensors were also readily arrayed; for example, it was extended from one valve to multiple valves to measure several valves' status, important for large-scale microfluidic systems that require real-time feedback to control each valve.

Fabrication of the soft strain sensors was improved for sensitivity from the previous protocol reported by Pegan et al (J. D. Pegan, J. Zhang, M. Chu, T. Nguyen, S.-J. Park, A. Paul, J. Kim, M. Bachman and M. Khine, Nanoscale, 2016, 8, 17295-17303). Specifically, the fabrication method involved tuning the thickness of the metals, improving the shrinking protocol, developing a soft, customized PDMS substrate, and introducing an encapsulation layer on the sensors.

Briefly, a layer of single-sided adhesive plastic shadow mask film was applied to a pre-stressed polystyrene sheet. The geometry of the mask was designed by laser etching, and then lifted off from the polystyrene sheet. Then a thickness-controlled magnetron sputter deposited 40 nm of Pt and 5 nm of Au onto the masked polystyrene sheet. The mask was removed and the polystyrene sheet was put in a convection oven set at 140 degree Celsius for 13 minutes. After the sheet shrank under heat, the sample was placed in a 5 mM 3-mercaptopropyl trimethoxysilane (MPTMS) ethanol solution for 2 hours. After rinsing away the excess MPTMS, the dried sample was covered with polydimethylsiloxane (PDMS), which had a mass ratio of 1:20:4.2 cure to base to dimethyl silicone fluid (PMX 200), and spin-coated at 800 RPM for 35 seconds. The sample was placed in vacuum to degas and was then cured at 60° C. overnight. The PDMS and the functional metal thin film was lifted off from the polystyrene by submerging the sample in a heated acetone bath. The PDMS and bonded metal thin film were further cleaned by additional acetone and toluene rinsing. In order to make the metal film electrically isolated from the environment, another layer of PDMS with the same composition as above was spun on the other side at 1000 RPM for 35 seconds. The sample was placed at room temperature for at least 48 hours to cure. After curing, the final sensor geometry was designed and laser etched through. The pad area of the sensor was sandwiched by two pieces of acrylic to reduce any potential movement to the pad and connection area. The 28-gauge silicone wires were connected to the pad with silver conductive epoxy (M.G. Chemical Ltd).

A Zaber linear actuator (Zaber Technologies Inc) was mounted onto a custom acrylic stage, and the entire system was placed within a custom acrylic box to prevent any possible environmental air flow that might affect the signal acquisition. The stage contained two parts: one part was stationary; the other part was able to slide on a track uniaxially. The driving side of the linear actuator was connected to the moving part of the stage. The pad side of the sensor was mounted on the stationary side of the stage while the other side of the sensor was clamped onto the moving portion of the stage. A Precision LCR Meter (Keysight Technologies E4980AL) was used to acquire resistance data of the sensor. A Labview based program was used to control movement of the stage and collect stage position data from the linear actuator and sensor resistance data from the LCR meter. The linear actuator applied 6 consecutive groups of micro-cycles of 5 μm, and each group contains 300 cycles. Then the entire process was repeated at different frequencies.

A 3 mm×15 mm×1.5 mm (w×l×h) acrylic piece was placed over the sensor trace area directly and a metal probe attached to a force gauge (Mark 10 M5-025). The force gauge was mounted on the test stand (Mark 10 ESM 303) and moved down at 20 μm/s speed until in contact with the acrylic piece. The test was repeated 5 times.

The microfluidic device contains two parts: channel and sensor. The channel was made with positive mold on a piece of PDMS (Young's modulus ˜2.6 MPa), and had a cross-sectional dimension of 50 μm×150 μm. The total length of the channel was 241.7 mm. For flow rates from 1 μl/min to 200 μl/min, the Reynolds number remained smaller than 40. Thus, the working range was always stable laminar flow, and there was no noise due to turbulence.

The thin film based piezoresistive sensor consists of two layers of PDMS with customized stiffness (Young's modulus ˜250 KPa) and one layer of wrinkled bimetallic thin film (platinum and gold). The total thickness of the layer was ˜100 μm. The metal film was sandwiched and firmly bonded in between PDMS layers to stay insulated and prevent from wearing and scratching. The polymer layers and wrinkled metal film deformed under stretching or compression; due to the brittle wrinkled structure of the metal film, micro-cracks formed. As more and larger cracks formed on the metal film, the electrical resistance increased.

The sensor was directly embedded at the bottom of the chip and served as the base of the channel. The pressure required to drive fluid flow deformed the channel. As the upper and side walls of the channel were about 10-fold stiffer than the bottom sensor wall, most of the deformation occurred on the sensor surface. The electrical resistance of the sensor increased due to the deformation described above.

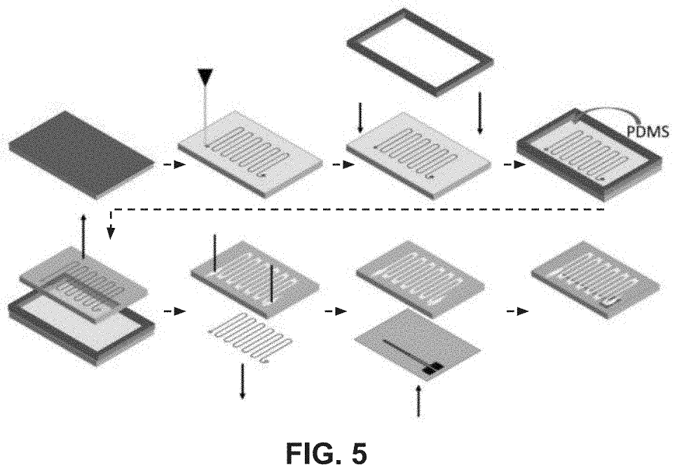

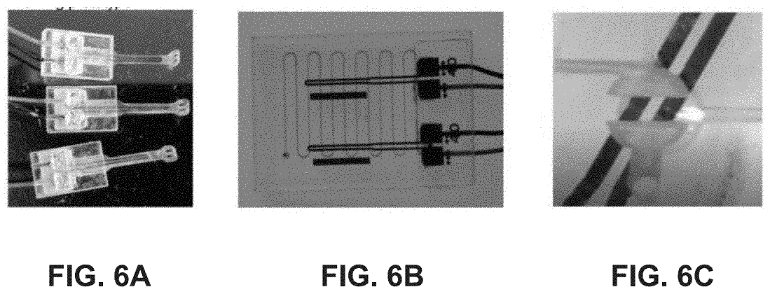

Referring now to FIG. 5, the present invention features a method for fabricating a stretchable sensor into a microfluidic channel to allow measurement of fluid directed through the said channel. The microfluidic device comprised two parts: sensor and channel. The sensor part followed the same procedure as regular sensor fabrication until curing of the encapsulation layer. After curing, the sensor was ready for plasma treatment. The channel device was fabricated through a traditional replica molding process (detailed flow chart is shown in FIG. 5). The positive mold was created by applying a layer of single-sided adhesive plastic film (Frisket Film from Grafix Art) on a piece of acrylic base. The shape of the channel was designed by laser etching the outline of the channel geometry. Excess plastic film outside the channel geometry was removed after laser etching. An acrylic well was adhered to the base to create a mold. 10:1 base to cure ratio of PDMS was poured into the mold, degassed for 20 minutes, and cured for 2 hours under 60° C. The PDMS channel device was removed from the positive mold and a biopsy punch was used to create an inlet and outlet. After cleaning both device and sensor with tape, the bottom side of the sensor and channel side of the device were placed in the plasma machine (Plasma Etch) and treated for 3 minutes. Then the sensor and device were placed with treated sides against each other and cured at 60° C. for over 2 hours for stronger bonding.

The outlet of the microfluidic chip was connected to a plastic pipeline and open to air. The inlet of the microfluidic chip was connected to a 3 ml syringe and controlled by a syringe pump. The syringe pump was programmed to deliver a specific flow rate to perform relevant working range, resolution, accuracy, repeatability and leaking tests. An inline pressure transducer (Omega PX 409) was connected to the syringe outlet via T-shaped connector. A Precision LCR Meter (Keysight Technologies) was used to acquire sensor resistance data.

Microfluidic valves and digital logic circuits were fabricated similarly to previous works. Microfluidic channels were machined into sheets of PMMA (Polymethyl methacrylate) using a CO2 laser (VLS 2.3, Universal Laser Systems) and devices were assembled by aligning and sandwiching the channel layers (channel had a width of 400 μm and depth of 400 μm, resistor had a width of 200 μm and depth of 200 μm) around a piece of sensor-embedded PDMS (˜600 μm thickness). The sensor was situated directly over the valve. For the flow control valve, a constant vacuum pressure of −85 kPa was applied to one side of the flow layer while a mass air flow meter (Zephyr HAF, Honeywell) was connected to the other side through 150 cm of 0.02″ ID Tygon microbore tubing. The valve was switched on and off with a period of 10 s and a control pressure of −85 kPa delivered via a computer-controlled miniature solenoid valve (S10, Pneumadyne, Plymouth, Minn.) while air flow measurements were acquired at a frequency of 90 Hz. Inverter gates were constructed similarly, leaving the input to the inverter open to room air and adding a pressure sensor (PX139, Omega) to the output of the gate. Pressure measurements were acquired at a frequency of 50 Hz. The oscillator pump consisted of a ring oscillator formed from three identical inverter gates connected in a ring and three liquid handling valves each connected to the output of an inverter. The flow rate of air from the peristaltic pump was measured by a hot wire anemometer (Zephyr HAF, Honeywell) connected to the output of the pump while images of the incident light reflected from a pump valve were acquired at 240 Hz by a camera (iPhone Xr, Apple Computer).The average pixel intensity of a region of interest over the valve was extracted and processed with a custom program written using OpenCV40.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A stretchable strain sensor (1) for detecting strain, pressure, deformation, stress, displacement, or a combination thereof, the sensor comprising:
a. a first soft polymer layer (100);
b. a wrinkled conductive layer (200) disposed on the first soft polymer layer (100); and
c. a second soft polymer layer (300) disposed on the wrinkled conductive layer (200);
wherein strain, pressure, deformation, stress, displacement, or a combination thereof applied to the strain sensor (1) causes the wrinkled conductive layer (200) to stretch and crack, creating resistance in the wrinkled conductive layer (200) and sending a signal based on the resistance;
wherein the first and second soft polymer layers have an elastic modulus ranging from about 225 kPa to about 275 kPa.

2. The stretchable strain sensor (1) of claim 1, wherein the strain sensor (1) is capable of detecting about 5 microns of linear displacement.

3. The stretchable strain sensor (1) of claim 1, wherein the stretchable strain sensor (1) is capable of measuring and sensing in vitro behavior of tissue and organs.

4. The stretchable strain sensor (1) of claim 1, wherein the stretchable strain sensor (1) is capable of measuring pressure and flow rate inside a channel of a microfluidic device.

5. The stretchable strain sensor (1) of claim 1, wherein the stretchable strain sensor (1) is capable of monitoring a status of a valve in a microfluidic device.

6. The stretchable strain sensor (1) of claim 1, wherein the first soft polymer layer (100) comprises PDMS, hydrogel, silicon-based polymers, polyurethane-based polymers, any polymer that can be molded, elastomers, or a combination thereof.

7. The stretchable strain sensor (1) of claim 1, wherein a soft polymer composition of the first soft polymer layer (100) and the second soft polymer layer (300) comprises polydimethylsiloxane (PDMS) having a mass ratio of about 1-4 cure to 15-20 base to 4-5 silicone fluid.

8. The stretchable strain sensor (1) of claim 1, wherein the wrinkled conductive layer (200) comprises one or more metals, one or more semiconductive materials, one or more nano-materials, one or more conductive polymers, one or more conductive particles embedded in a polymer, or a combination thereof, wherein the wrinkled conductive layer (200) has a thickness of about 100 nm.

9. The stretchable strain sensor (1) of claim 1, wherein the second soft polymer layer (300) comprises PDMS, hydrogel, silicon-based polymers, polyurethane-based polymers, any polymer that can be molded, elastomers, or a combination thereof.

10. The stretchable strain sensor (1) of claim 1, wherein the stretchable strain sensor (1) can be tuned to detect a wider range of forces through the use of PDMS fluid.

11. The stretchable strain sensor (1) of claim 1, wherein the strain sensor (1) is capable of returning to a resting state from strain, pressure, deformation, stress, displacement, or a combination thereof in about 5-10 ms.

12. A method for measuring strain, pressure, deformation, stress, displacement, or a combination thereof using a stretchable strain sensor (1), the method comprising:
a. providing the stretchable strain sensor (1), the sensor comprising:
a first soft polymer layer (100);
a wrinkled conductive layer (200) disposed on the first soft polymer layer (100); and
a second soft polymer layer (300) disposed on the wrinkled conductive layer (200);
wherein strain, pressure, deformation, stress, displacement, or a combination thereof applied to the strain sensor (1) causes the wrinkled conductive layer (200) to stretch and crack, creating resistance in the wrinkled conductive layer (200) and sending a signal based on the resistance;
wherein the first and second soft polymer layers have an elastic modulus ranging from about 225 to 275 kPa;
b. applying strain, pressure, deformation, stress, displacement, or a combination thereof to the strain sensor (1);
c. stretching and cracking, by the wrinkled conductive layer (200), in response to the strain, pressure, deformation, stress, displacement, or combination thereof of the strain sensor (1);
d. generating, by the wrinkled conductive layer (200), resistance as a result of stretching and cracking; and
e. sending a signal based on the resistance generated by the wrinkled conductive layer (200).

13. The method of claim 12, wherein the method is utilized to measure and sense in vitro behavior of tissue and organs.

14. The method of claim 12, wherein the method is utilized to measure pressure and flow rate inside a channel of a microfluidic device, monitor a status of a valve in a microfluidic device, or a combination thereof.

15. A method for fabricating a stretchable strain sensor (1) into a microfluidic channel to allow measurement of strain, pressure, deformation, stress, displacement, or a combination thereof in the microfluidic channel, the method comprising:
a. applying heat to a conductive material layer to cause shrinkage in order to produce a wrinkled conductive layer (200);
b. preparing and tuning a polymer composition to have an elastic modulus to 225 to 275 kPa, thereby producing a soft polymer composition
c. applying a first layer (100) comprising the soft polymer composition to a first side of the wrinkled conductive layer (200); and
d. applying a second layer (300) comprising the soft polymer composition to a second side of the wrinkled conductive layer (200) such that the wrinkled conductive layer (200) is disposed between the first soft polymer layer (100) and the second soft polymer layer (300).

16. The method of claim 15, wherein the strain sensor (1) is capable of detecting about 5 microns of linear displacement.

17. The method of claim 15, wherein the soft polymer composition comprises PDMS, hydrogel, silicon-based polymers, polyurethane-based polymers, any polymer that can be molded, elastomers, or a combination thereof.

18. The method of claim 15, wherein the soft polymer composition comprises polydimethylsiloxane (PDMS) having a mass ratio of about 1-4 cure to 15-20 base to 4-5 silicone fluid.

19. The method of claim 15, wherein the wrinkled conductive layer (200) comprises one or more metals, one or more semiconductive materials, one or more nano-materials, one or more conductive polymers, one or more conductive particles embedded in a polymer, or a combination thereof, wherein the wrinkled conductive layer (200) has a thickness of about 100 nm.

20. The method of claim 15, wherein the strain sensor (1) is capable of returning to a resting state from strain, pressure, deformation, stress, displacement, or a combination thereof in about 5 to 10 ms.
